(19) United States
(12) Patent Application Publication
(10) Pub. No.: US 2018/0065900 A1
(43) Pub. Date: Mar. 8, 2018

(54) Process of Alkylation of Isobutane with Olefins

(71) Applicant: Alvega Technologies, Inc., Dover, DE (US)

(72) Inventors: Oleg I. PARPUTC, Kirishy (RU); Petr A. ZERNOV, Pcheva (RU)

(21) Appl. No.: 15/693,742

(22) Filed: Sep. 1, 2017

Related U.S. Application Data
(60) Provisional application No. 62/385,004, filed on Sep. 8, 2016.

(51) Int. Cl. C07C 2/62; B01J 23/30; B01J 21/06; B01J 35/02

(52) U.S. Cl. CPC C07C 2/62; B01J 23/30; C07C 2523/30; B01J 35/023; C07C 2521/06; B01J 21/066

(57) ABSTRACT

The process for producing an alkylate is intended for use in the oil refining and hydrocarbon process industry. The process comprises reacting isobutane with an alkylation agent (an olefin) in the presence of a solid acidic catalyst and a C4+ solvent. Thus, a system is obtained, wherein the solid acidic catalyst is suspended in a solvent and the suspension is maintained by passing the feed stream through the catalyst-solvent system, thereby providing a uniform catalyst distribution throughout the entire volume and enabling the reaction to be carried out at low pressures.

### RELATED APPLICATIONS

This application claims priority to U.S. Provisional Application No. 62/385,004, filed Sep. 8, 2016, which is incorporated herein by reference in its entirety.

### FIELD OF THE INVENTION

The invention relates to a process for the production of branched paraffinic hydrocarbons by alkylation of isobutane with olefinic hydrocarbons in the presence of solid catalysts. This method can be used in the oil refining and petrochemical industries.

### BACKGROUND OF THE INVENTION

Processes for the production of an alkylate from isobutane and olefins are fairly well covered in the literature.

At this moment, the most applicable methods comprise using liquid acids HF and H₂SO₄. These methods, however, have a number of significant drawbacks. First, these acids are toxic and highly corrosive.

Recently, methods for alkylation on solid catalysts or solid superacids have been widely described. Despite the obvious advantages (no toxic emissions, no corrosion reactions), these methods have certain drawbacks. Therefore, for carrying out the process of alkylation on solid catalysts, it is necessary to generate pressure in order to carry out the process in the liquid phase. Given that the process on solid catalysts is carried out at temperatures of 70-100° C., the pressure must be substantial, i.e. at least 20 atmospheres. It is well known, however, that in terms of thermodynamics, the alkylation reaction is preferably carried out at low temperatures. Higher temperatures result in decreased process selectivity and formation of undesired by-products such as oligomerizate and cracked gas. The formation of by-products leads to rapid deactivation of the catalyst, which requires frequent regeneration. Considering that, as a rule, the processes on solid catalysts are carried out in fixed-bed reactors, the number of such reactors must be at least 3 to ensure the continuity of the process.

In addition, alkylation systems in the suspended layer and suspension-type systems are being developed recently.

US2012/0230882 A1 describes a system for alkylation of isobutane with olefin to make an alkylate. This system is a column in which the catalyst is suspended in liquid isobutane and the alkylation agent—olefin is supplied in a liquid and/or gaseous state. The suspension is supported by circulating the gas phase formed during the reaction. The process is conducted at temperatures of 60-80° C. and a pressure of about 11 atmospheres.

The referenced method suffers the following drawbacks. Considering that the boiling point of isobutane is lower than that of, e.g. butenes, the butenes will not create a vapor stream when they enter a column of liquid isobutane, but rather will condense. Correspondingly, the catalyst distribution throughout the volume will be impaired and, consequently, the conversion and process efficiency will decrease. To form a substantial vapor stream, it is necessary that cracking side reactions take place along with the desired alkylation reaction. Accordingly, the process selectivity and the yield of alkylate decreases. These drawbacks substantially limit the application of the referenced method.

### SUMMARY OF THE INVENTION

The present invention provides a process for producing an alkylate by reacting isobutane with olefins in a suspension reactor. The inventive process comprises:
preparing a suspension of a solid acidic catalyst in a solvent, where the solvent is a C4+ hydrocarbon;
conducting the reaction by supplying the feed stream (isobutane+olefin) into the prepared suspension in a gaseous state, wherein the stable suspension and the uniform catalyst distribution are maintained by a substantial flow of the feed stream and no mechanical stirring is required;
drawing off a part of the suspension from the reactor and separating the resulting mixture into an alkylate and a solvent-catalyst system;
returning the solvent-catalyst system to the suspension reactor,

wherein a boiling point of the C4+solvent is higher than or equal to a boiling point of the lowest boiling point hydrocarbon of the feed stream and wherein the reaction temperature is maintained above the boiling point of the lowest boiling point hydrocarbon of the feed stream, but below the boiling point of the C4+ solvent.

In addition to the main flow diagram, a part of the solvent-catalyst system is provided to the regeneration section, where regeneration of the catalyst is carried out by known methods (reduction and/or oxidation).

Also, the present invention provides for supplying fresh catalyst and solvent.

### BRIEF DESCRIPTION OF DRAWINGS

FIG. 1—shows a process flow diagram.

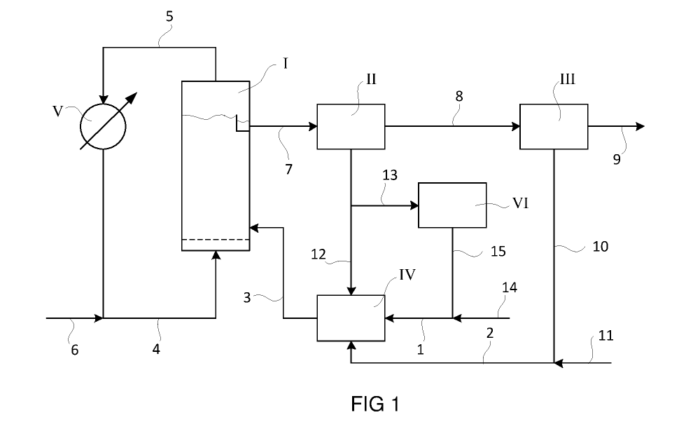

### DETAILED DESCRIPTION OF THE PREFERRED EMBODIMENTS

As used herein, the articles including “a” and “an” when used in the Specification or in a claim, are understood to mean one or more of what is described or claimed.

As used herein, the terms “include”, “includes”, “including”, “comprise”, “comprises”, “comprising” are meant to be non-limiting.

Catalyst 1 is mixed with a C4+ solvent 2 by any known method in mixing unit IV to form a suspension 3. In addition, the suspension 3 can be prepared directly in the reaction zone I. The catalyst is in the form of solid particles of about 20-500 μm in size. Due to the use of this size fraction of the catalyst, the available surface of active sites increases significantly as compared with massive catalysts and therefore a much smaller amount of the catalyst per unit of reactants is required. In one embodiment, it is possible to use, as a solid acidic catalyst, promoted and non-promoted Y and/or BEA type zeolites. In another embodiment, it is possible to use solid superacids as a solid acidic catalyst. Preferably, sulfated and/or tungsten zirconium oxides are used as solid superacid catalysts. Elements and/or oxides thereof providing stability and/or hydride transfer (Fe, Mn, Ti, Ni, Pt, Pd, Re, Sn, Ga, La, Ce) can also further promote these oxides.

As solvent 2, a C4+ hydrocarbon with a boiling point higher than or equal to the boiling point of the lowest boiling point hydrocarbon of the feed stream 4 is used. Of the wide range of hydrocarbons, n-paraffin hydrocarbons are the most preferred, as the least reactive. Non-limiting examples of suitable hydrocarbon solvents include n-butane, n-pentane, n-hexane. Also, as solvent 2, an alkylate or a mixture of alkylate and a C4+ hydrocarbon can be used. The main requirement for such mixtures and the solvent as a whole is that its boiling point is higher than or equal to the boiling point of the lowest boiling point component of the feed stream. This requirement provides that the lowest boiling point component of the feedstock, upon entering the solvent-catalyst reaction system, will provide the suspension system with the necessary stirring due to its boiling.

A feed stream 4 prepared by any known method and consisting of isobutane and an olefin is supplied into the suspension 3. Non-limiting examples of olefins include ethylene, propylene, butene-2. The mixture is prepared by any known method, such that the isobutane/olefin molar ratio at the entering to reaction zone I was equal to from about 10:1 to about 500:1, preferably from about 100:1 to about 400:1. The temperature in reaction zone I is maintained at about 20-100° C. and is determined by the type of catalyst used. Most preferably, the reaction is carried out at a temperature of about 20-50° C. which results in the reduction in the required pressure when using low-boiling solvents. In addition, low temperatures, as is known to one of ordinary skills in the art, are thermodynamically favorable for the formation of the target products—trimethylpentanes.

Unreacted components of mixture 5 leave the reaction zone and are sent to condensation/cooling system V, and then mixed with feed stream 6, provided that an isobutane/olefin molar ratio in feed stream 4 at the entering to reaction zone I is maintained, as described above.

The reaction products together with a part of the suspension 7 are drawn off for separation. The separation of the solid catalyst and the liquid phase is carried out in unit II by any known method (evaporation, centrifugation, use of hydrocyclones, etc.). Then a mixture of solvent and alkylate 8 is sent to unit III for separation by any known method, e.g., by rectification. Alkylate 9 is sent to the tank farm, and solvent 10 is recycled to prepare the suspension, which solvent can be premixed with fresh solvent 11 if necessary.

After separation 12, the catalyst is sent to prepare the suspension, wherein a part of the catalyst 13 is drawn off and replaced with a fresh one 14 or regenerated one 15. The removed part of the catalyst 13 is sent to unit VI for regeneration by any known method (boil-out with isobutane, oxidation in airflow or reduction with hydrogen at higher temperatures, etc.). Regenerated catalyst 15 is used to prepare the suspension.

The inventors have surprisingly found out that when the process of alkylation of isobutane with olefins is carried out as set forth above, the lifetime of the non-modified/non-promoted catalysts drastically rises as compared to that under the conditions of a customary tubular reactor. That surprising finding makes it possible for the process to be carried out at a reduced internal paraffin/olefin molar ratio, which results in a lower amount of the catalyst (active component) required in the process. This effect is illustrated in Example 4.

### EXAMPLES

The term “Time on Stream” in all Examples means a period of time from the start of the reaction to the moment when olefin conversion starts to drop down.

### Example 1

5 grams of sulfated zirconia oxide were first suspended in 10 ml of n-hexane with a gaseous nitrogen stream in a jacketed reactor. The suspension was heated to 30° C., the temperature was controlled by thermocouples along the height of the reactor. Then nitrogen was replaced with a feed stream with an isobutane/butene-2 molar ratio of 200:1. The flow rate of the gas mixture was set at 100 g/h. Drawing-off of the suspension-product mixture was set at 2 g/h on the alkylate basis. The system pressure was 1 absolute atmosphere. The product was analyzed by gas chromatography with a flame ionization detector on a 50 m long capillary column.

The results are shown in Table 1.

### Example 2

5 grams of sulfated zirconia oxide were first suspended in 10 ml of n-pentane with a gaseous nitrogen stream in a jacketed reactor. The suspension was heated to 30° C.; the temperature was controlled by thermocouples along the height of the reactor. Then nitrogen was replaced with a feed stream with an isobutane/butene-2 molar ratio of 200:1. The flow rate of the gas mixture was set at 100 g/h. Drawing-off of the suspension-product mixture was set at 2 g/h on the alkylate basis. The system pressure was 1.5 absolute atmosphere. The product was analyzed by gas chromatography with a flame ionization detector on a 50 m long capillary column.

The results are shown in Table 1.

### Example 3

Spent catalyst from Example 1 was regenerated by oxidation in static air at 400° C. for 1 hour. Then, the experiment of Example 1 was repeated.

The results are shown in Table 1.

### Example 4

5 grams of the tungsten zirconia oxide were first suspended in 10 ml of n-heptane with a gaseous nitrogen stream in a jacketed reactor. The suspension was heated to 50° C., the temperature was controlled by thermocouples along the height of the reactor. Then nitrogen was replaced with a feed stream with an isobutane/butene-2 molar ratio of 20:1. The flow rate of the gas mixture was set at 100 g/h. Drawing-off of the suspension-product mixture was set at 2 g/h on the alkylate basis. The system pressure was 1 absolute atmosphere. The product was analyzed by gas chromatography with a flame ionization detector on a 50 m long capillary column.

The results are shown in Table 1.

TABLE 1

| Characteristic | Example 1 | Example 2 | Example 3 | Example 4 |
|---|---|---|---|---|
| Olefin conversion, % wt | 99.5 | 99.2 | 99.6 | 99.1 |
| Yield of alkylate C8-C9, | 79.6 | 79.5 | 81.1 | 79.6 |
| % wt | | | | |
| Content of | 35.6 | 33.9 | 35.9 | 34.2 |
| trimethylpentane in | | | | |
| alkylate, % wt | | | | |
| Time on Stream, hours | 10 | 10 | 10 | 5 |

What is claimed is:

1. A process for producing an alkylate by reacting isobutane with an olefin in the presence of a solid acidic catalyst in a suspension, the process comprising:
suspending the solid acidic catalyst in a C4+ hydrocarbon solvent;
supplying a feed stream in an amount sufficient to maintain the suspension in a stable state, wherein the feed stream comprises isobutane and olefin, and wherein a boiling point of the C4+ hydrocarbon solvent is higher than a boiling point of a lowest boiling point hydrocarbon of the feed stream; and
carrying out the process at a temperature above the boiling point of the lowest boiling point hydrocarbon of the feed stream, but below the boiling point of the C4+ hydrocarbon solvent.

2. The process of claim 1, wherein the solid acidic catalyst has a particle size of about 20-500 μm.

3. The process of claim 1, wherein the solid acidic catalyst is selected from promoted or non-promoted Y and/or BEA type zeolites or solid superacids.

4. The process of claim 3, wherein a solid superacid of the solid superacids is selected from sulfated zirconium oxide and/or tungsten zirconium oxide.

5. The process of claim 1, wherein a molar ratio of the isobutene to olefin in the feed stream is from about 10:1 to about 500:1.

6. The process of claim 1, wherein the C4+ hydrocarbon solvent comprises an n-paraffin and/or an alkylate.

7. The process of claim 6, wherein said n-paraffin is selected from n-butane, n-pentane and n-hexane.

8. The process of claim 1, wherein the temperature is from about 20° C. to about 50° C.

9. The process of claim 1, wherein carrying out the process occurs at a pressure from about 1 to about 10 absolute atmospheres.

10. The process of claim 1, wherein the feed stream is in a gaseous state.
